(19) United States
(12) Patent Application Publication
(10) Pub. No.: US 2024/0130932 A1
(43) Pub. Date: Apr. 25, 2024

(54) CALCIUM SILICATE-BASED HYDRAULIC CEMENT TO FORM A COMPOSITE MATERIAL HAVING REINFORCING PROPERTIES

(71) Applicant: SEPTODONT OU SEPTODONT SAS ou SPECIALITES SEPTODONT, Saint-Maur-des-Fossés (FR)

(72) Inventors: Mounir DJOUDI, Saint-Maur-des-Fossés (FR); Gilles RICHARD, Saint-Maur-des-Fossés (FR); Lauren ARTAUD, Saint-Maur-des-Fossés (FR)

(73) Assignee: SEPTODONT OU SEPTODONT SAS ou SPECIALITES SEPTODONT, Saint-Maur-des-Fossés (FR)

(21) Appl. No.: 18/546,663

(22) PCT Filed: Mar. 4, 2022
(86) PCT No.: PCT/EP2022/055649

§ 371 Date: Aug. 15, 2023

(30) Foreign Application Priority Data
Mar. 4, 2021 (EP) 21305261.6

(51) Int. Cl. A61K 6/17; A61K 6/853; A61K 6/873

(52) U.S. Cl. CPC A61K 6/17; A61K 6/853; A61K 6/873

(57) ABSTRACT

Hydraulic cements and hardened materials obtained after hydration of the hydraulic cements, especially cements useful in the medical field such as dental cements. In particular, a hydraulic cement including calcium silicate particles and calcium carbonate particles having specific particles sizes. The hydration of the hydraulic cement enables to provide a hardened composite material with reinforcing properties, having calcium silicate particles dispersed in a solid dispersant phase including calcium silicate hydrates (CSH) and porous intergranular areas including insoluble calcium carbonate particles having a d₅₀ granulometry ranging from 1 nm to 1500 nm.

### FIELD OF INVENTION

The present invention relates to the field of hydraulic cements and hardened materials obtained after hydration of said hydraulic cements. Especially, the present invention deals with a hydraulic cement comprising calcium silicate particles and calcium carbonate particles of specific particles sizes. The hydration of the hydraulic cement of the invention enables to provide a hardened composite material having reinforcing mechanical properties. The present invention also refers to the method for manufacturing said composite material, and its uses in the medical field or in the non-therapeutical cement field.

### BACKGROUND OF INVENTION

High performance cementitious materials prepared from hydraulic calcium silicate cement, such as Portland cement, are increasingly finding their use both in medical and non-therapeutical fields (such as construction), worldwide.

Whatever the fields, there is always a need for providing hardened cementitious materials featuring reinforced mechanical properties such as for example higher compressive strength. Moreover, there is always a need for providing processes for manufacturing said materials, which are less time consuming and therefore, more economical.

The Applicant evidenced that the introduction, in a calcium silicate-based hydraulic cement, of calcium carbonate particles with specific particles' size populations, enables to provide upon hydration a hardened cementitious material having reinforcing mechanical properties. Without wishing to be bound by any theory, the Applicant submits that the specific particles' size distributions of the calcium carbonate used in the hydraulic cement of the invention leads to the formation, upon hydration, of porous intergranular areas with submicrometric calcium carbonate particles, which provides improved mechanical properties for the hardened cementitious material.

The Applicant has also evidenced that when the manufacturing of a hardened cementitious material comprises at least one mixing step by vibration of a powder phase comprising calcium silicate particles and calcium carbonate particles, with an aqueous phase, it enables to provide a hardened material with the same type of reinforcing mechanical properties. Without wishing to be bound by any theory, the Applicant submits that the mixing step by vibration provides a self-grinding of at least a part of the calcium carbonate particles contained in the hydraulic cement, leading to calcium carbonate particles populations having the specific particles sizes according to the invention, so that the final hardened cementitious material comprises reinforcing intergranular areas with submicrometric calcium carbonate particles.

### SUMMARY

The present invention thus provides a hydraulic cement comprising:
15% to 98% in weight of the total weight of the cement of calcium silicate particles, wherein 50% in volume of the total volume of calcium silicate particles have a size ranging from 1 μm to 10 μm; preferably from 1 μm to 8 μm; and
0.5% to 85% in weight of the total weight of the cement of calcium carbonate particles, wherein:
   10% in volume of the total volume of the calcium carbonate particles have a size of less than 0.59 μm;
   50% in volume of the total volume of the calcium carbonate particles have a size of less than 2.5 μm, and
   40% in volume of the total volume of the calcium carbonate particles have a size ranging from 2.5 μm to 20 μm.

In one embodiment, the calcium carbonate particles are such that:
10% in volume of the total volume of the calcium carbonate particles have a size of less than 0.55 μm;
50% in volume of the total volume of the calcium carbonate particles have a size of less than 1.5 μm, and
40% in volume of the total volume of the calcium carbonate particles have a size ranging from 1.5 μm to 5.5 μm.

In one embodiment, the calcium silicate particles are selected from tricalcium silicate (C3S), dicalcium silicate (C2S) and any combinations thereof; preferably the calcium silicate particles are tricalcium silicate particles.

In one embodiment, the calcium silicate particles are in a Portland cement or in a mineral trioxide aggregate (MTA).

In one embodiment, the hydraulic cement further comprises at least one additive, preferably selected from set accelerators, radiopacifiers, pigments, pH stabilizing agents, fillers, texturing/thickening agents, water-reducing agents and mixtures thereof.

In one embodiment, the radiopacifier is selected from zirconium oxide, bismuth oxide, cerium oxide, barium sulphate, calcium tungstate, titanate dioxide, ytterbium oxide and mixtures thereof; preferably the radiopacifier is zirconium oxide.

The invention also provides a method for manufacturing a hydraulic cement according to the invention, comprising mixing
15% to 98% in weight of the total weight of the cement of calcium silicate particles, wherein 50% in volume of the total volume of calcium silicate particles have a size ranging from 1 μm to 10 μm; preferably from 1 μm to 8 μm; and
0.5% to 85% in weight of the total weight of the cement of calcium carbonate particles, wherein:
   10% in volume of the total volume of the calcium carbonate particles have a size of less than 0.59 μm;
   50% in volume of the total volume of the calcium carbonate particles have a size of less than 2.5 μm, and
   40% in volume of the total volume of the calcium carbonate particles have a size ranging from 2.5 μm to 20 μm.

The invention also provides another method for manufacturing a hydraulic cement according to the invention, comprising
at least one mixing step of a powder phase comprising:
   15% to 98% in weight of the total weight of the cement of calcium silicate particles, wherein 50% in volume of the total volume of calcium silicate particles have a size ranging from 1 μm to 10 μm; preferably from 1 μm to 8 μm; and
   0.5% to 85% w/w of calcium carbonate particles, wherein
      10% in volume of the total volume of the calcium carbonate particles have a size of less than 1 μm;
      50% in volume of the total volume of the calcium carbonate particles have a size of less than 5 μm, and
      40% in volume of the total volume of the calcium carbonate particles have a size ranging from 5 μm to 30 μm;
and a simultaneous and/or sequential vibration step leading to a hydraulic cement according to the invention.

In one embodiment, the vibration step is implemented with a vibration frequency ranging from 1 rpm to 15 000 rpm; preferably ranging from 1000 rpm to 6 000 rpm; more preferably ranging from 3 000 rpm to 5 000 rpm; and during a vibration time ranging from 1 s to 3600 s; preferably from 1 s to 60 s; more preferably during 30 s.

In one embodiment, the mixing of the powder phase includes a mixing by three-dimensional motion.

The invention further provides a method for manufacturing a composite material, comprising at least one mixing step of a hydraulic cement according to the invention, with an aqueous phase; in a mass ratio of the hydraulic cement to the aqueous phase ranging from 2 to 4.5.

The invention also relates to a composite material obtained by the process according to the invention, comprising
a solid dispersant phase comprising or consisting of calcium silicate hydrates (CSH);
calcium silicate particles dispersed in the solid dispersant phase;
a porous intergranular area located between said calcium silicate particles; said porous intergranular area comprising insoluble calcium carbonate particles having a d₅₀ granulometry ranging from 1 nm to 1500 nm; preferably from 1 nm to 1000 nm.

The invention further provides a kit for producing a composite material, said kit comprising:
a powder phase comprising the hydraulic cement according to the invention; and
a liquid aqueous phase;
wherein the weight ratio of the powder phase present in the kit to the liquid aqueous phase present in the kit ranges from 2 to 5; preferably from 2.5 to 4.0.

The invention also relates to the use of a hydraulic cement according to the invention, to manufacture a composite material as reinforcing material in the non-therapeutical cement field.

The invention also relates to the hydraulic cement according to the invention, for use in the medical field, preferably in the dental or the orthopedic field, to form a restorative and/or filling material.

### Definitions

In the present invention, the following terms have the following meanings:
“Additive” refers to any substance added, preferably in a low amount, in a composition for improving its physicochemical properties depending on its use. The additive may for example be selected from radiopacifiers (such as zirconium oxide), setting accelerators (such as calcium oxide, calcium carbonate, calcium chloride), pigments (such as iron oxides), water reducing agents (such as modified polycarboxylates), texturing agents, pH stabilizing agents, surfactants, fillers, and mixtures thereof.
“Aqueous” refers to any compound or composition comprising water and/or moisture.
“Calcium silicate” refers to compounds that can be produced by reacting calcium oxide and silica in various ratios. According to one embodiment, the expression “calcium silicate” refers to compounds made of calcium and silicate, preferably selected from tricalcium silicate, dicalcium silicate or any mixtures thereof; more preferably tricalcium silicate C3S (of formula Ca₃SiO₅), dicalcium silicate C2S (of formula Ca₂SiO₄), or any mixtures thereof.
“Calcium silicate particle”: refers to an assembly comprising one or more calcium silicate compounds. The terms “calcium silicate particle” also include assemblies consisting of one or more calcium silicate compounds.
“Calcium silicate hydrates” refers to any products resulting from the hydration of calcium silicate. According to one embodiment, the terms “calcium silicate hydrates” mean a compound of formula (I):

mCaO·nSiO₂·pH₂O
in which n and m independently range from 1 to 3, and p ranges from 3 to 6. According to one embodiment, the terms “calcium silicate hydrates” refer to the products resulting from the hydration of dicalcium silicate and/or tricalcium silicate. According to one embodiment, the terms “calcium silicate hydrates” refer to a compound of formula (I) as defined above in which m equals 3, n equals 2 and p equals 3. According to one embodiment, the terms “calcium silicate hydrates” also include calcium hydroxide (Ca(OH)₂). In the present invention, the expression “porous calcium silicate hydrates (p-CSH)” means that the calcium silicate hydrates form a matrix with pores; preferably with a pore size ranging from more than 0 nm to less than 1 μm. According to one embodiment, the porous calcium silicate hydrates is a CSH matrix with pores having a pore size higher than 50 nm, preferably ranging from 51 nm to 1 μm; preferably ranging from 51 nm to 100 nm. According to one embodiment, the porous calcium silicate hydrates is a CSH matrix with pores having a pore size ranging from higher than 2 nm to 50 nm. According to one embodiment, the porous calcium silicate hydrates is a CSH matrix with pores having a pore size ranging from more than 0 nm to 2 nm. In the present invention, the expression “dense calcium silicate hydrates (d-CSH)” means that the calcium silicate hydrates form a matrix less porous than the porous calcium silicate hydrates as defined above. According to one embodiment, the expression “dense calcium silicate hydrates (d-CSH)” means that the calcium silicate hydrates form a matrix without any pores.
“Composite material” refers to any material comprising or consisting of the association of at least two non-miscible components for which the resulting physical and/or chemical properties are different from those of these components taken individually.
“Dental cement” refers to any composition suitable for restorative dentistry that acts as an adhesive to hold together the casting to the tooth structure.
“d10 granulometry”: means that 10% of the particles have a mean diameter less than said value. According to one embodiment, the d₁₀ size is measured by laser diffraction.
“d50 granulometry”: means that 50% of the particles have a mean diameter less than said value. According to one embodiment, the d₅₀ size is measured by laser diffraction.
“d90 granulometry”: means that 90% of the particles have a mean diameter less than said value. According to one embodiment, the d₉₀ size is measured by laser diffraction.
“Dispersant phase”: refers to a chemical medium in which are dispersed particles.
“Hardened dental material”: refers to a material suitable for dental applications that is under a solid form. A “hardened dental filling material” especially refers to a hardened dental material suitable for filling dental restoration.
“Hydraulic cement”: refers to a cement able to self-harden when contacted with water. According to one embodiment, the cement is a dental hydraulic cement. According to one embodiment, the cement is a non-therapeutical hydraulic cement.
“Laser diffraction analysis”: refers to a technique using diffraction patterns of a laser beam passed through a particulate object for determining its size.
“Pigment”: refers to any coloring chemical compound that may be natural or synthetic, mineral or organic.
“Porous”: refers to a mineral compound comprising mesopores, micropores and/or macropores. The pores of the porous material typically have a pore size ranging from more than 0 nm to 1 μm; for instance from 2 nm to 1 μm.
“Portland cement”: refers to a hydraulic material comprising at least two-thirds by mass of calcium silicates, (3 CaO·SiO₂, and 2 CaO·SiO₂) as main component, and comprising additional compounds including aluminum- and/or iron-containing clinker phases (for example, tricalcium aluminate and tetracalcium aluminoferrite). The expression “Portland cement” includes all the Portland cement compositions well-known by the skilled artisan such as those defined by the European EN 197 norm, and by the International ASTM C150 norm.
“Pozzolanic material” refers, according to ASTM C125, to a natural or artificial siliceous or siliceous and aluminous material which, in itself, possesses little or no cementitious value but which will, in finely divided form and in the presence of water, reacts chemically with calcium hydroxide at ordinary temperature to form compounds possessing cementitious properties. An artificial pozzolanic material may comprise or consisting of, for instance, an industrial by-product such as fly ash, silica fume from silicon smelting, highly reactive metakaolin, slag, burned organic matter residues rich in silica such as rice husk ash, calcined clay, or any mixture thereof. A natural pozzolanic material may comprise or consist of, for instance, volcanic ashes, pumices, zeolites, diatomaceous earths and any mixture thereof. According to one embodiment, the at least one pozzolanic material is selected from the group consisting of fly ash, silica fume, metakaolin, slag, and rice husk ash.
“Radiopacifying agent” or “radiopacifier”: refers to a substance added to a material in order to make it opaque, especially to make it visible under X-ray imaging.
“Reinforcing properties”: refers to any compound able to improve physical properties of a material, preferably to enhance the mechanical properties of a material such as the compressive strength for example.
“Silica fume”: refers to an amorphous (non-crystalline) polymorph of silicon dioxide, silica. Silica fume is an ultrafine powder collected as a by-product of the silicon and ferrosilicon alloy production and consists of spherical particles with an average particle diameter of 150 nm. Silica fume is also known as microsilica and its CAS number is 69012-64-2, its EINECS number is 273-761-1.
“Water-reducing agent”: refers to a substance able to improve the rheological properties of a composition. Especially, the “water-reducing agent” may be a plastifying or fluidifying agent.

Further, in the present invention, when referring to a range, the following is meant: “ranging from X to Y” means that X and Y are included in the range; “ranging from more than X, up to Y” means that X is not included in the range while Y is included in the range; and “less than X” means that the range includes X or lower values.

### DETAILED DESCRIPTION

Hydraulic Cement

This invention relates to a hydraulic cement, especially a calcium silicate-based hydraulic powder cement. The hydraulic cement of the invention enables to form, upon hydration, a hardened composite material. Due to the composition of the hydraulic cement of the invention, the resulting hardened composite material present improved properties, especially improves mechanical properties, in particular with respect to resistance to compression and a lesser porosity, compared to reference compositions.

The hydraulic cement of the invention comprises:
calcium silicate particles, preferably selected from tricalcium silicate, dicalcium silicate, Portland cement, mineral trioxide aggregate (MTA) and any combinations thereof, more preferably tricalcium silicate; and
calcium carbonate particles.

The hydraulic cement of the invention is anhydrous.

Calcium Silicate Particles

The hydraulic cement of the invention thus comprises calcium silicate particles.

In one embodiment, the calcium silicate particles are selected from tricalcium silicate (C3S), dicalcium silicate (C2S) and any combinations thereof; preferably the calcium silicate particles are tricalcium silicate particles. In another embodiment, the calcium silicate particles are in a Portland cement or in a mineral trioxide aggregate (MTA).

According to one embodiment, the hydraulic cement comprises calcium silicate particles in an amount ranging from 10% to 98% by weight of the total weight of the cement, preferably from 15% to 85%, preferably from 20% to 85%, more preferably from 50% to 81%.

According to one embodiment, 50% in volume of the total volume of calcium silicate particles present in the cement of the invention have a size ranging from 1 μm to 10 μm; preferably from 1 μm to 8 μm.

According to one embodiment, the d₁₀ granulometry of calcium silicate particles in the hydraulic cement of the invention ranges from more than 0 μm to 2 μm; preferably from 0.1 μm to 2 μm. According to one embodiment, the d₁₀ granulometry of calcium silicate particles in the cement is 0.1, 0.2, 0.3, 0.4, 0.5, 0.6, 0.7, 0.8, 0.9 or 1 μm. According to one embodiment, the d₁₀ granulometry ranges as defined above are determined by laser diffraction.

According to one embodiment, the d₅₀ granulometry of calcium silicate particles in the hydraulic cement of the invention ranges from more than 0 μm to 10 μm; preferably from 1 μm to 8 μm; more preferably from 3 μm to 8 μm or from 0.5 μm to 3 μm. According to one embodiment, the d₅₀ granulometry of calcium silicate particles in the cement is 1, 2, 3, 4, 5, 6, 7, 8, 9 or 10 μm. According to one embodiment, the d₅₀ granulometry ranges as defined above are determined by laser diffraction.

According to one embodiment, the d₉₀ granulometry of calcium silicate particles in the hydraulic cement of the invention ranges from more than 0 μm to 20 μm; preferably from 1 μm to 10 μm; more preferably from 1 μm to 7 μm. According to one embodiment, the d₉₀ granulometry of calcium silicate particles in the cement is 1, 2, 3, 4, 5, 6, 7, 8, 9, 10, 11, 12, 13, 14, 15, 16, 17, 18, 19 or 20 μm. According to one embodiment, the 4:150 granulometry in the mixture ranges as defined above are determined by laser diffraction.

Calcium Carbonate Particles

The hydraulic cement of the invention also comprises calcium carbonate particles.

According to one embodiment, the hydraulic cement comprises calcium carbonate particles in an amount ranging from 0.5% to 85% by weight of the total weight of the cement, preferably from 5% to 50%, preferably from 5% to 20.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.59 μm; preferably of less than 0.55 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.55 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
50% in volume of the total volume of the calcium carbonate particles have a size of less than 2.5 μm; preferably of less than 2.35 μm; preferably of less than 1.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
50% in volume of the total volume of the calcium carbonate particles have a size of less than 1.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.59 μm; preferably of less than 0.55 μm; and/or
50% in volume of the total volume of the calcium carbonate particles have a size of less than 2.5 μm; preferably of less than 2.35 μm; preferably of less than 1.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.55 μm; and/or
50% in volume of the total volume of the calcium carbonate particles have a size of less than 1.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
40% in volume of the total volume of the calcium carbonate particles have a size ranging from 2.5 μm to 20 μm; preferably ranging from 2.35 μm to 20 μm; preferably from 1.5 μm to 20 μm; preferably from 1.5 μm to 5.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
40% in volume of the total volume of the calcium carbonate particles have a size ranging from 1.5 μm to 5.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.59 μm; preferably of less than 0.55 μm; and/or
40% in volume of the total volume of the calcium carbonate particles have a size ranging from 2.5 μm to 20 μm; preferably ranging from 2.35 μm to 20 μm; preferably from 1.5 μm to 20 μm; preferably from 1.5 μm to 5.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
10% in volume of the total volume of the calcium carbonate particles have a size of less than 0.55 μm; and/or
40% in volume of the total volume of the calcium carbonate particles have a size ranging from 1.5 μm to 5.5 μm.

According to one embodiment, the hydraulic cement comprises calcium carbonate particles in an amount ranging from 0.5% to 85% by weight of the total weight of the cement, preferably from 5% to 50%, preferably from 5% to 20, wherein the population of calcium carbonate particles in the hydraulic cement of the invention is as follows:
20% or 30% in volume of the total volume of the calcium carbonate particles have a size of less than 1.50 μm; preferably of less than 1.0 μm;

According to one embodiment, the population of calcium carbonate particles in the hydraulic cement of the invention is as follows:
50% in volume of the total volume of the calcium carbonate particles have a size of less than 2.5 μm; preferably of less than 2.35 μm; preferably of less than 1.5 μm; and/or
40% in volume of the total volume of the calcium carbonate particles have a size ranging from 2.5 μm to 20 μm; preferably ranging from 2.35 μm to 20 μm; preferably from 1.5 μm to 20 μm; preferably from 1.5 μm to 5.5 μm.

According to one embodiment, the population of calcium carbonate particles in the hydraulic cement of the invention is as follows:
50% in volume of the total volume of the calcium carbonate particles have a size of less than 1.5 μm; and/or
40% in volume of the total volume of the calcium carbonate particles have a size ranging from 1.5 μm to 5.5 μm.

According to one embodiment, the population of calcium carbonate particles in the hydraulic cement of the invention is as follows:
10% in volume of the total volume of the calcium carbonate particles have a size of less than 0.59 μm; preferably of less than 0.55 μm;
50% in volume of the total volume of the calcium carbonate particles have a size of less than 2.5 μm; preferably of less than 2.35 μm; preferably of less than 1.5 μm; and/or
40% in volume of the total volume of the calcium carbonate particles have a size ranging from 2.5 μm to 20 μm; preferably ranging from 2.35 μm to 20 μm; preferably from 1.5 μm to 20 μm; preferably from 1.5 μm to 5.5 μm.

According to one embodiment, the population of calcium carbonate particles in the hydraulic cement of the invention is as follows:
10% in volume of the total volume of the calcium carbonate particles have a size of less than 0.55 μm;
50% in volume of the total volume of the calcium carbonate particles have a size of less than 1.5 μm; and/or
40% in volume of the total volume of the calcium carbonate particles have a size ranging from 1.5 μm to 5.5 μm.

According to one embodiment, calcium carbonate particles in the hydraulic cement of the invention have a d₁₀ granulometry ranging from 1 nm to 1000 nm; preferably from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, or from 1 nm to 100 nm. According to one embodiment, calcium carbonate particles in the mixture have a d₁₀ granulometry of 590 nm or 550 nm.

According to one embodiment, calcium carbonate particles in the hydraulic cement of the invention have a d₅₀ granulometry ranging from 1 nm to 5 000 nm; preferably from 1 nm to 4000 nm, from 1 nm to 3000 nm, from 1 nm to 2000 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, or from 1 nm to 100 nm. According to one embodiment, calcium carbonate particles in the hydraulic cement of the invention have a d₅₀ granulometry ranging from 2500 nm to 3000 nm. According to one embodiment, calcium carbonate particles in the mixture have a d₅₀ granulometry of 2500 nm, 2350 nm or 1500 nm.

According to one embodiment, calcium carbonate particles in the hydraulic cement of the invention have a d₉₀ granulometry ranging from 1 nm to 30 000 nm; preferably from 1 nm to 29 000 nm, from 1 nm to 27 000 nm, from 1 nm to 26 000 nm, from 1 nm to 25 000 nm, from 1 nm to 24 000 nm, from 1 nm to 23 000 nm, from 1 nm to 22 000 nm, from 1 nm to 21 000 nm, from 1 nm to 20 000 nm, from 1 nm to 19 000 nm, from 1 nm to 18 000 nm, from 1 nm to 17 000 nm, from 1 nm to 16 000 nm, from 1 nm to 15 000 m, from 1 nm to 14 000 nm, from 1 nm to 13 000 nm, from 1 nm to 12 000 nm, from 1 nm to 11 000 nm, from 1 nm to 10 000 nm, from 1 nm to 9 000 nm, from 1 nm to 8 000 nm, from 1 nm to 7 000 nm, from 1 nm to 6 000 nm, from 1 nm to 5 000 nm, from 1 nm to 4000 nm, from 1 nm to 3000 nm, from 1 nm to 2000 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm or from 1 nm to 100 nm. According to one embodiment, calcium carbonate particles in the cement have a d₉₀ granulometry ranging from 25000 nm to 30000 nm. According to one embodiment, calcium carbonate particles in the mixture have a d₉₀ granulometry of 20000 nm or 5500 nm.

Other Components

According to one embodiment, the hydraulic cement of the invention further comprises at least one additive; preferably selected from setting accelerator, radiopacifiers, pigments, pH stabilizing agents, fillers, texturing/thickening agents, water-reducing agents, surfactants, and mixtures thereof.

According to one embodiment, the filler is a pozzolanic material; preferably selected from the group consisting of fly ash, silica fume, metakaolin, slag, and rice husk ash.; more preferably is silica fume.

According to one embodiment, the radiopacifier is selected from zirconium oxide, bismuth oxide, cerium oxide, barium sulphate, calcium tungstate, titanate dioxide, ytterbium oxide and mixtures thereof. In a specific embodiment, the radiopacifier is zirconium oxide.

According to one embodiment, the setting accelerator is calcium carbonate, calcium oxide, calcium phosphate, sodium bicarbonate, calcium lactate, calcium chloride or mixtures thereof. According to one embodiment, the setting accelerator is calcium carbonate, calcium oxide or mixtures thereof. According to one embodiment, the setting accelerator is calcium chloride.

According to one embodiment, the pigments may be iron oxides.

According to one embodiment, the water-reducing agent is selected from glenium, polynaphtalene sulfonate, modified polycarboxylate.

According to one embodiment, the texturing agents may be for example selected from silica, povidone (also named polyvinylpyrrolidone), cellulose or derivatives thereof such as methylcellulose, hydroxypropylcellulose and hydroxyethylcellulose, polymers such as acrylamide/sodium acryloyldimethyltaurate copolymer isohexadecane and hydroxyethyl acrylate/sodium acryloyl dimethyl taurate copolymer, mineral fillers, fumed silica (hydrophilic and/or hydrophobic), xanthan gum, or mixtures thereof.

According to one embodiment, the pH stabilizing agent is a mineral acid or an organic acid.

According to one embodiment, the surfactant is a polysorbate.

According to one embodiment, the hydraulic cement of the invention comprises at least one additive in an amount ranging from 0% to 60% in weight to the total weight of the cement; preferably from 2% to 50%; more preferably from 2% to 35%. According to one embodiment, the hydraulic cement of the invention comprises at least one additive in an amount ranging from 0% to 30% in weight to the total weight of the cement; preferably from 1% to 25%; more preferably from 1% to 18%.

According to one embodiment, the hydraulic cement of the invention comprises from 0% to 40% of radiopacifier in weight to the total weight of said cement; preferably from 2 to 35%, from 5 to 35%, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35%. According to one embodiment, the hydraulic cement of the invention comprises from 0% to 20% of radiopacifier in weight to the total weight of said cement; preferably from 1 to 18%, from 2 to 18%.

According to one embodiment, the radiopacifier presents a hardness of more than 3.5 preferably more than 5 even more preferably more than 8 on the Mohs scale. According to a specific embodiment, the radiopacifier is Zirconium dioxide and the hydraulic cement of the invention comprises from 0% to 40% of radiopacifier in weight to the total weight of said cement; preferably from 2 to 35%, from 5 to 35% in weight to the total weight of the cement composition.

Composite Material

This invention also relates to a composite material, preferably a hardened composite material. According to one embodiment, the composite material is a hardened cementitious material. According to one embodiment, the composite material is a solid material. According to one embodiment, the composite material of the invention results from the hydration a hydraulic cement. According to one embodiment, the composite material of the invention results from the hydration of a hydraulic cement comprising a calcium silicate compound; preferably selected from tricalcium silicate, dicalcium silicate, Portland cement, mineral trioxide aggregate (MTA) and any combinations thereof; more preferably the composite material of the invention results from the hydration a hydraulic cement comprising tricalcium silicate. According to one embodiment, the composite material of the invention results from the hydration a hydraulic cement comprising a calcium silicate compound as defined above, under the form of particles.

According to one embodiment, the composite material of the invention results from the hydration of the hydraulic cement according to the invention.

According to one embodiment, the composite material of the invention results from the hydration of a hydraulic cement comprising or consisting of a calcium silicate compound, calcium carbonate and a pozzolanic material, preferably selected from the group consisting of fly ash, silica fume, metakaolin, slag, and rice husk ash.; more preferably is silica fume.

According to one embodiment, the hydration of a hydraulic cement leads to a hardened material. According to one embodiment, the composite material of the invention is a hardened composite material, preferably obtained by the hydration of a hydraulic cement; more preferably obtained by the hydration of a hydraulic cement comprising a calcium silicate compound as defined above.

According to one embodiment, the (hardened) composite material comprises or consists of a dispersant phase, preferably a solid dispersant phase, and calcium silicate particles dispersed in said dispersant phase. According to one embodiment, the (hardened) composite material comprises or consists of a solid dispersant phase and insoluble calcium silicate particles dispersed in said solid dispersant phase. According to one embodiment, the (hardened) composite material comprises or consists of a solid dispersant phase and non-hydrated calcium silicate particles dispersed in said solid dispersant phase. In the present invention, the expression “non-hydrated calcium silicate particles” means that the calcium silicate particles did not react with water or moisture.

According to one embodiment, the (hardened) composite material further comprises at least one additive; preferably selected from setting accelerator, radiopacifiers, pigments, pH stabilizing agents, fillers, texturing/thickening agents, water-reducing agents, surfactant, and mixtures thereof.

According to one embodiment, the filler is a pozzolanic material; preferably selected from the group consisting of fly ash, silica fume, metakaolin, slag, and rice husk ash.; more preferably is silica fume. According to one embodiment, the radiopacifier is selected from zirconium oxide, bismuth oxide, cerium oxide, barium sulphate, calcium tungstate, titanate dioxide, ytterbium oxide and mixtures thereof. In a specific embodiment, the radiopacifier is zirconium oxide. According to one embodiment, the setting accelerator is calcium carbonate, calcium oxide, calcium phosphate, sodium bicarbonate, calcium lactate, calcium chloride or mixtures thereof. According to one embodiment, the setting accelerator is calcium carbonate, calcium oxide or mixtures thereof. According to one embodiment, the setting accelerator is calcium chloride. According to one embodiment, the pigments may be iron oxides. According to one embodiment, the water-reducing agent is selected from glenium, polynaphtalene sulfonate, modified polycarboxylate. According to one embodiment, the texturing agents may be for example selected from silica, povidone (also named polyvinylpyrrolidone), cellulose or derivatives thereof such as methylcellulose, hydroxypropylcellulose and hydroxyethylcellulose, polymers such as acrylamide/sodium acryloyldimethyltaurate copolymer isohexadecane and hydroxyethyl acrylate/sodium acryloyl dimethyl taurate copolymer, mineral fillers, fumed silica (hydrophilic and/or hydrophobic), xanthan gum, or mixtures thereof. According to one embodiment, the pH stabilizing agent is a mineral acid or an organic acid. According to one embodiment, the surfactant is a polysorbate.

According to one embodiment, the (hardened) composite material comprises at least one additive in an amount ranging from 0% to 60% in weight to the total weight of the composite material; preferably from 2% to 50%; more preferably from 2% to 35%. According to one embodiment, the (hardened) composite material comprises at least one additive in an amount ranging from 0% to 30% in weight to the total weight of the composite material; preferably from 1% to 25%; more preferably from 1% to 18%.

According to one embodiment, the (hardened) composite material comprises from 0 to 40% of radiopacifier in weight to the total weight of said (hardened) composite material; preferably from 2 to 35%, from 5 to 35%, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35%. According to one embodiment, the (hardened) composite material comprises from 0 to 20% of radiopacifier in weight to the total weight of said (hardened) composite material; preferably from 1 to 18%, from 2 to 18%.

Dispersant Phase

According to one embodiment, the dispersant phase is a solid dispersant phase. According to one embodiment, the dispersant phase, preferably the solid dispersant phase, comprises or consists of at least one product of the hydration of a calcium silicate compound; preferably selected from tricalcium silicate, dicalcium silicate, Portland cement, mineral trioxide aggregate (MTA) and any combinations thereof; more preferably the solid dispersant phase comprises or consists of at least one product of the hydration of a hydraulic cement comprising tricalcium silicate.

According to one embodiment, the product(s) of the hydration of a calcium silicate compound may be a calcium silicate hydrate (CSH), calcium oxide (CaO) and/or a calcium hydroxide (Ca(OH)₂). According to one embodiment, the solid dispersant phase comprises or consists of at least one product of the hydration of a calcium silicate compound selected from calcium silicate hydrate (CSH), calcium oxide (CaO), calcium hydroxide (Ca(OH)₂) and mixtures thereof.

According to one embodiment, the calcium silicate hydrate (CSH) results from the hydration of tricalcium silicate (C3S) and/or dicalcium silicate (C2S).

According to one embodiment, the solid dispersant phase comprises or consists of at least one calcium silicate hydrate (CSH); preferably a calcium silicate hydrate of the following formula (I):

mCaO·nSiO₂·pH₂O

in which n and m independently range from 1 to 3 and p ranges from 3 to 6; preferably m equals 3, n equals 2 and p equals 3.

According to one embodiment, the solid dispersant phase comprises or consists of dense calcium silicate hydrates (d-CSH) as defined above.

According to one embodiment, the solid dispersant phase comprises or consists of porous calcium silicate hydrates (p-CSH) as defined above.

According to one embodiment, the solid dispersant phase comprises pores with a pore size ranging from more than 0 nm to 1 μm, preferably from 2 nm to 1 μm, more preferably from 2 nm to 100 nm, more preferably from 2 nm to 50 nm, more preferably from 2 nm to 20 nm, in particular from 8 nm to 15 nm.

According to one embodiment, the dispersant phase further comprises a pozzolanic material, preferably selected from the group consisting of: fly ash, silica fume, metakaolin, slag and rice husk ash; more preferably silica fume.

Dispersed Calcium Silicate Particles

According to one embodiment, the d₁₀ granulometry of calcium silicate particles in the composite material ranges from more than 0 μm to 2 μm; preferably from 0.1 μm to 2 μm. According to one embodiment, the d₁₀ granulometry of calcium silicate particles in the composite material is 0.1, 0.2, 0.3, 0.4, 0.5, 0.6, 0.7, 0.8, 0.9 or 1 μm. According to one embodiment, the d₁₀ granulometry of non-hydrated calcium silicate particles in the composite material ranges from more than 0 μm to 2 μm; preferably from 0.1 μm to 2 μm. According to one embodiment, the d₁₀ granulometry of non-hydrated calcium silicate particles in the composite material is 0.1, 0.2, 0.3, 0.4, 0.5, 0.6, 0.7, 0.8, 0.9 or 1 μm. According to one embodiment, the d₁₀ granulometry ranges as defined above are determined by laser diffraction.

According to one embodiment, the 4:150 granulometry of calcium silicate particles in the composite material ranges from more than 0 μm to 10 μm; preferably from 1 μm to 8 μm; more preferably from 3 μm to 8 μm or from 0.5 μm to 3 μm. According to one embodiment, the 4:150 granulometry of calcium silicate particles in the composite material is 1, 2, 3, 4, 5, 6, 7, 8, 9 or 10 μm. According to one embodiment, the 4:150 granulometry of non-hydrated calcium silicate particles in the composite material ranges from more than 0 μm to 10 μm; preferably from 1 μm to 8 μm; more preferably from 3 μm to 8 μm or from 0.5 μm to 3 μm. According to one embodiment, the 4:150 granulometry of non-hydrated calcium silicate particles in the composite material is 1, 2, 3, 4, 5, 6, 7, 8, 9 or μm. According to one embodiment, the 4:150 granulometry ranges as defined above are determined by laser diffraction.

According to one embodiment, the d₉₀ granulometry of calcium silicate particles in the composite material ranges from more than 0 μm to 20 μm; preferably from 1 μm to 10 μm; more preferably from 1 μm to 7 μm. According to one embodiment, the d₉₀ granulometry of calcium silicate particles in the composite material is 1, 2, 3, 4, 5, 6, 7, 8, 9, 10, 11, 12, 13, 14, 15, 16, 17, 18, 19 or 20 μm. According to one embodiment, the d₉₀ granulometry of non-hydrated calcium silicate particles in the composite material ranges from more than 0 μm to 20 μm; preferably from 1 μm to 10 μm; more preferably from 1 μm to 7 μm. According to one embodiment, the d₉₀ granulometry of non-hydrated calcium silicate particles in the composite material is 1, 2, 3, 4, 5, 6, 7, 8, 9, 10, 11, 12, 13, 14, 15, 16, 17, 18, 19 or 20 μm. According to one embodiment, the d₅₀ granulometry ranges as defined above are determined by laser diffraction.

According to one embodiment, the amount of calcium silicate particles in the composite material ranges from 5% wt. to 65% wt., preferably 8% wt. to 60% wt., more preferentially 10% wt. to 35% wt., by the total weight of said (hardened) composite material. According to one embodiment, the amount of non-hydrated calcium silicate particles ranges from 5% wt. to 65% wt., preferably 8% wt. to 60% wt., more preferentially 10% wt. to 35% wt., by the total weight of said (hardened) composite material.

According to one embodiment, the amount of calcium silicate particles in the composite material is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35 or 35% wt. by the total weight of said (hardened) composite material. According to one embodiment, the amount of non-hydrated calcium silicate particles in the composite material is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35 or 35% wt. by the total weight of said (hardened) composite material.

Intergranular Area

According to one embodiment, the (hardened) composite material comprises one or more intergranular area(s), preferably located between and/or around the particles of the calcium silicate compound as defined above. According to one embodiment, the (hardened) composite material comprises one or more intergranular area(s) located between and/or around non-hydrated calcium silicate particles dispersed in the dispersant phase as defined above.

According to one embodiment, the intergranular area(s) embed, partially or totally, one or more calcium silicate particles as defined above. According to one embodiment, the intergranular area(s) embed, partially or totally, one or more non-hydrated calcium silicate particles as defined above.

According to one embodiment, the intergranular area comprises or consists of calcium carbonate (CaCO₃), preferably under the form of particles and/or aggregates. According to one embodiment, the intergranular area comprises or consists of insoluble calcium carbonate (CaCO₃), preferably under the form of particles and/or aggregates.

According to one embodiment, the intergranular area further comprises at least one product of the hydration of a calcium silicate compound as defined above; preferably at least one product of the hydration of a calcium silicate compound selected from tricalcium silicate, dicalcium silicate and mixtures thereof; more preferably at least one product of the hydration of tricalcium silicate. According to one embodiment, the intergranular area further comprises one or more calcium silicate hydrates (CSH).

According to one embodiment, the intergranular area comprises or consists of at least one calcium silicate hydrate (CSH) and calcium carbonate (CaCO₃), preferably under the form of particles and/or aggregates. According to one embodiment, the intergranular area comprises or consists of at least one calcium silicate hydrate (CSH) and insoluble calcium carbonate (CaCO₃), preferably under the form of particles and/or aggregates.

According to one embodiment, the calcium silicate hydrate (CSH) in the intergranular area is porous calcium silicate hydrate (p-CSH) as defined above. According to one embodiment, the calcium silicate hydrate (CSH) in the intergranular area is dense calcium silicate hydrate (d-CSH) as defined above. According to one embodiment, the intergranular area comprises or consists of porous calcium silicate hydrate(s) (p-CSH) and calcium carbonate (CaCO₃). According to one embodiment, the intergranular area comprises or consists of dense calcium silicate hydrate(s) (d-CSH) and calcium carbonate (CaCO₃).

According to one embodiment, the intergranular area is porous. According to one embodiment, the intergranular area comprises mesopores, micropores and/or macropores. According to one embodiment, the pore size is determined by mercury intrusion porosimetry (MIP). Alternatively, the pore size may be determined by Transmission Electron Microscopy (TEM).

The porosity of the intergranular area may vary in a wide range. In some embodiments the porosity of the intergranular area is quite low. A quite low porosity of the intergranular area contributes to reinforcing the mechanical properties of the composite material.

According to one embodiment, the (insoluble) calcium carbonate particles in the composite material of the invention have a d₁₀ granulometry ranging from 1 nm to 500 nm; preferably from 1 nm to 400 nm, from 1 nm to 300 nm, from 1 nm to 200 nm, from 1 nm to 100 nm, from 1 nm to 90 nm, from 1 nm to 80 nm, from 1 nm to 70 nm, from 1 nm to 60 nm, or from 1 nm to 50 nm. According to one embodiment, the (insoluble) calcium carbonate particles have a d₁₀ granulometry ranging from 400 nm to 500 nm. According to one embodiment, the (insoluble) calcium carbonate particles have a d₁₀ granulometry of 440 nm.

According to one embodiment, the (insoluble) calcium carbonate particles in the composite material of the invention have a d₅₀ granulometry ranging from 1 nm to 1500 nm; preferably from 1 nm to 1400 nm, from 1 nm to 1300 nm, from 1 nm to 1200 nm, from 1 nm to 1100 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, from 1 nm to 100 nm, from 1 nm to 50 nm, or from 1 nm to 10 nm. According to one embodiment, the (insoluble) calcium carbonate particles have a d₅₀ granulometry ranging from 1000 nm to 1200 nm. According to one embodiment, the (insoluble) calcium carbonate particles have a d₅₀ granulometry of 1100 nm.

According to one embodiment, the (insoluble) calcium carbonate particles in the composite material of the invention have a d₉₀ granulometry ranging from 1 nm to 5 000 nm; preferably from 1 nm to 4000 nm, from 1 nm to 3000 nm, from 1 nm to 2000 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, from 1 nm to 100 nm, from 1 nm to 50 nm, or from 1 nm to nm. According to one embodiment, the (insoluble) calcium carbonate particles have a d₉₀ granulometry of 3000 nm to 3600 nm. According to one embodiment, the (insoluble) calcium carbonate particles have a d₉₀ granulometry of 3550 nm.

According to one embodiment, the (insoluble) calcium carbonate particles in the composite material of the invention have size distribution as follows:
at least 10% in volume of the total volume of the calcium carbonate particles have a size of less than 0.59 μm; preferably of less than 0.55 μm; and/or
at least 50% in volume of the total volume of the calcium carbonate particles have a size of less than 2.5 μm; preferably of less than 2.35 μm; preferably of less than 1.5 lim.

According to one embodiment, the (insoluble) calcium carbonate particles in the composite material of the invention have size distribution as follows:

10% in volume of the total volume of the calcium carbonate particles have a size of less than 0.59 μm; preferably of less than 0.55 μm;
50% in volume of the total volume of the calcium carbonate particles have a size of less than 2.5 μm; preferably of less than 2.35 μm; preferably of less than 1.5 μm; and
40% in volume of the total volume of the calcium carbonate particles have a size ranging from 2.5 μm to 20 μm; preferably ranging from 2.35 μm to 20 μm; preferably from 1.5 μm to 20 μm; preferably from 1.5 μm to 5.5 μm.

According to one embodiment, the amount of the (insoluble) calcium carbonate particles in the composite material of the invention ranges from more than 0% wt. to 20% wt., preferably from 1% wt. to 15% wt., more preferably from 1% wt. to 7% wt. by the total weight of said composite material. According to one embodiment, the amount of the (insoluble) calcium carbonate particles in the composite material of the invention is 1, 2, 3, 4, 5, 6, 7, 8, 9, 10, 11, 12, 13, 14, 15, 16, 17, 18, 19 or 20% wt., by the total weight of said composite material.

According to one embodiment, the intergranular area further comprises a pozzolanic material, preferably selected from the group consisting of: fly ash, silica fume, metakaolin, slag, and rice husk ash; more preferably silica fume.

According to one embodiment, the amount of the pozzolanic material in the composite material of the invention ranges from more than 0% wt. to 20% wt., preferably from 1% wt. to 15% wt., more preferably from 1% wt. to 7% wt. by the total weight of said composite material. According to one embodiment, the amount of the pozzolanic material in the composite material of the invention is 1, 2, 3, 4, 5, 6, 7, 8, 9, 10, 11, 12, 13, 14, 15, 16, 17, 18, 19 or 20% wt., by the total weight of said composite material.

Specific Composite Material

According to one embodiment, the (hardened) composite material comprises or consists of:
a dispersant phase, preferably a solid dispersant phase, comprising or consisting of calcium silicate hydrates (CSH);
calcium silicate particles, preferably tricalcium silicate particles, dispersed in the dispersant phase; and
one or more intergranular area(s) comprising or consisting of insoluble calcium carbonate particles;
said insoluble calcium carbonate particles having a 4:150 granulometry ranging from 1 nm to 1500 nm; preferably from 1 nm to 1000 nm.

According to one embodiment, the (hardened) composite material comprises or consists of:
a dispersant phase, preferably a solid dispersant phase comprising or consisting of calcium silicate hydrates (CSH);
calcium silicate particles, preferably tricalcium silicate particles, dispersed in the solid dispersant phase; and
one or more porous intergranular area(s), located between said calcium silicate particles; said porous intergranular area comprising insoluble calcium carbonate particles having a d₅₀ granulometry ranging from 1 nm to 1500 nm; preferably from 1 nm to 1000 nm.

According to one embodiment, the (hardened) composite material comprises or consists of:
a dispersant phase, preferably a solid dispersant phase, comprising or consisting of calcium silicate hydrates (CSH);
calcium silicate particles, preferably tricalcium silicate particles, dispersed in the dispersant phase; and
one or more intergranular area(s) comprising or consisting of at least one pozzolanic material and insoluble calcium carbonate particles;
said insoluble calcium carbonate particles having a 4:150 granulometry ranging from 1 nm to 1500 nm; preferably from 1 nm to 1000 nm.

According to one embodiment, the (hardened) composite material comprises or consists of:
a dispersant phase, preferably a solid dispersant phase, comprising or consisting of calcium silicate hydrates (CSH);
from 5% wt. to 65% wt. of calcium silicate particles, preferably tricalcium silicate particles, dispersed in the dispersant phase, by the total weight of the composite material; and
one or more intergranular area(s) comprising or consisting of insoluble calcium carbonate particles;
said insoluble calcium carbonate particles having a 4:150 granulometry ranging from 1 nm to 1500 nm; preferably from 1 nm to 1000 nm;

According to one embodiment, the amount of the insoluble calcium carbonate particles ranges from 1% wt. to 20% wt. by the total weight of said composite material.

According to one embodiment, the (hardened) composite material comprises or consists of:
a dispersant phase, preferably a solid dispersant phase, comprising or consisting of calcium silicate hydrates (CSH);
from 5% wt. to 65% wt. of calcium silicate particles, preferably tricalcium silicate particles, dispersed in the dispersant phase, by the total weight of the composite material; and
one or more intergranular area(s) comprising or consisting of at least one pozzolanic material and insoluble calcium carbonate particles;
said insoluble calcium carbonate particles having a 4:150 granulometry ranging from 1 nm to 1500 nm; preferably from 1 nm to 1000 nm;
optionally,
the amount of the at least one pozzolanic material ranges from 1% wt. to 15% wt. by the total weight of said composite material; and
the amount of the insoluble calcium carbonate particles ranges from 1% wt. to 5% wt. by the total weight of said composite material.

According to one embodiment, the (hardened) composite material comprises or consists of:
a solid dispersant phase comprising or consisting of calcium silicate hydrates (CSH);
from 5% wt. to 65% wt. of non-hydrated tricalcium silicate particles dispersed in the dispersant phase, by the total weight of the composite material; and
one or more intergranular area(s) comprising or consisting of insoluble calcium carbonate particles;
said insoluble calcium carbonate particles having a d₅₀ granulometry ranging from 1 nm to 1500 nm; preferably from 1 nm to 1000 nm;
optionally, the amount of the insoluble calcium carbonate particles ranges from 1% wt. to 20% wt. by the total weight of said composite material.

According to one embodiment, the (hardened) composite material comprises or consists of:
from 5% to 65%, preferably from 8% to 60%, more preferentially from 10% to 35% by weight with respect to the total weight of the (hardened) composite material, of calcium silicate particles, said calcium silicate particles preferably being chosen from the group comprising tricalcium silicate, dicalcium silicate, Portland cement, mineral trioxide aggregates (MTA) and/or mixtures thereof,
from 1% to 20%, preferably from 2% to 15% by weight with respect to the total weight of the (hardened) composite material, of calcium carbonate, preferably insoluble calcium carbonate particles; and
from more than 0% to 50%, preferably from 1% to 40% by weight with respect to the total weight of the (hardened) composite material, of mCaO·nSiO₂·pH₂O (CSH) in which m and n, each independently, vary from 1 to 3 and p varies from 3 to 6.

According to one embodiment, the (hardened) composite material comprises or consists of:
from more than 0% to 50%, preferably from 1% to 40% by weight with respect to the total weight of the (hardened) composite material, of a dispersant phase comprising or consisting of mCaO·nSiO₂·pH₂O (CSH) in which m and n, each independently, vary from 1 to 3 and p varies from 3 to 6;
from 5% to 65%, preferably from 8% to 60%, more preferentially from 10% to 35% by weight with respect to the total weight of the (hardened) composite material, of calcium silicate particles, said calcium silicate particles preferably being chosen from the group comprising tricalcium silicate, dicalcium silicate, Portland cement, mineral trioxide aggregates (MTA) and/or mixtures thereof; said calcium silicate particles being dispersed in the dispersant phase; and
from 1% to 20%, preferably from 2% to 15% by weight with respect to the total weight of the (hardened) composite material, of calcium carbonate, preferably insoluble calcium carbonate particles; said insoluble calcium carbonate particles be located in one or more intergranular area(s) located between and/or around the calcium silicate particles.

According to one embodiment, the (hardened) composite material has a compressive strength measured 24 h after mixing the powder phase (or the hydraulic cement) and the aqueous phase, ranging from more than 0 MPa to 400 MPa, preferably from 10 MPa to 300 MPa, more preferably from 50 MPa to 250 MPa.

Process for Manufacturing the Hydraulic Cement

The invention also relates to methods for manufacturing the hydraulic cement according to the invention.

First Method

Especially, the invention provides a first method for manufacturing a hydraulic cement according to the invention, comprising mixing the powders of calcium silicate and calcium carbonate, both having the targeted particles sizes distributions.

In one embodiment, the first method for manufacturing a hydraulic cement according to the invention comprises mixing:
15% to 98% in weight of the total weight of the cement of calcium silicate particles, wherein 50% in volume of the total volume of calcium silicate particles have a size ranging from 1 μm to 10 μm; preferably from 1 μm to 8 μm; and
0.5% to 85% in weight of the total weight of the cement of calcium carbonate particles, wherein:
   10% in volume of the total volume of the calcium carbonate particles have a size of less than 0.59 μm;
   50% in volume of the total volume of the calcium carbonate particles have a size of less than 2.5 μm, and
   40% in volume of the total volume of the calcium carbonate particles have a size ranging from 2.5 μm to 20 μm.

The mixing can be performed by any method known by one skilled in the art. In particular, gentle mixing in an apparatus providing three-dimensional motion (e.g. Turbula) can be used.

Second Method

The invention also provides a second method for manufacturing a hydraulic cement according to the invention, comprising mixing by vibration the powders of calcium silicate and calcium carbonate, having particles sizes distributions different from the final targeted particles sizes distributions. Especially, in this method, calcium carbonate particles of larger particles sizes are used and the method of mixing by vibration enables to reach the targeted particles' size distributions.

In one embodiment, the second method for manufacturing a hydraulic cement according to invention comprises at least one mixing step of a powder phase comprising:
15% to 98% in weight of the total weight of the cement of calcium silicate particles, wherein 50% in volume of the total volume of calcium silicate particles have a size ranging from 1 μm to 10 μm; preferably from 1 μm to 8 μm; and
0.5% to 85% w/w of calcium carbonate particles, wherein
   10% in volume of the total volume of the calcium carbonate particles have a size of less than 1 μm;
   50% in volume of the total volume of the calcium carbonate particles have a size of less than 5 μm, and
   40% in volume of the total volume of the calcium carbonate particles have a size ranging from 5 μm to 30 μm;
and vibration step that is simultaneous and/or sequential to the mixing step, leading to the hydraulic cement composition according to the invention.

In this second method, the granulometries of the calcium silicate and calcium carbonate powders subjected to the mixing step may also be as defined below.

According to one embodiment, the powder of the mixing step further comprises adding a radiopacifier presenting a hardness of more than 3.5 preferably more than 5 even more preferably more than 8 on the Mohs scale. According to a specific embodiment, the radiopacifier is Zirconium dioxide and the powder phase of the mixing step comprises from 0% to 40% of radiopacifier in weight to the total weight of said cement; preferably from 2 to 35%, from 5 to 35% in weight to the powder phase composition.

Calcium Silicate Granulometry

According to one embodiment, the d₁₀ granulometry of calcium silicate particles in the mixture ranges from more than 0 μm to 2 μm; preferably from 0.1 μm to 2 μm. According to one embodiment, the d₁₀ granulometry of calcium silicate particles in the mixture is 0.1, 0.2, 0.3, 0.4, 0.5, 0.6, 0.7, 0.8, 0.9 or 1 μm. According to one embodiment, the d₁₀ granulometry of non-hydrated calcium silicate particles in the mixture ranges from more than 0 μm to 2 μm; preferably from 0.1 μm to 2 μm. According to one embodiment, the d₁₀ granulometry of non-hydrated calcium silicate particles in the mixture is 0.1, 0.2, 0.3, 0.4, 0.5, 0.6, 0.7, 0.8, 0.9 or 1 μm. According to one embodiment, the d₁₀ granulometry ranges as defined above are determined by laser diffraction.

According to one embodiment, the 4:150 granulometry of calcium silicate particles in the mixture ranges from more than 0 μm to 10 μm; preferably from 1 μm to 8 μm; more preferably from 3 μm to 8 μm or from 0.5 μm to 3 μm. According to one embodiment, the 4:150 granulometry of calcium silicate particles in the mixture is 1, 2, 3, 4, 5, 6, 7, 8, 9 or 10 μm. According to one embodiment, the 4:150 granulometry of non-hydrated calcium silicate particles in the mixture ranges from more than 0 μm to 10 μm; preferably from 1 μm to 8 μm; more preferably from 3 μm to 8 μm or from 0.5 μm to 3 μm. According to one embodiment, the 4:150 granulometry of non-hydrated calcium silicate particles in the mixture is 1, 2, 3, 4, 5, 6, 7, 8, 9 or 10 μm. According to one embodiment, the 4:150 granulometry ranges as defined above are determined by laser diffraction.

According to one embodiment, the d₉₀ granulometry of calcium silicate particles in the mixture ranges from more than 0 μm to 20 μm; preferably from 1 μm to 10 μm; more preferably from 1 μm to 7 μm. According to one embodiment, the d₉₀ granulometry of calcium silicate particles in the mixture is 1, 2, 3, 4, 5, 6, 7, 8, 9, 10, 11, 12, 13, 14, 15, 16, 17, 18, 19 or 20 μm. According to one embodiment, the d₉₀ granulometry of non-hydrated calcium silicate particles in the mixture ranges from more than 0 μm to 20 μm; preferably from 1 μm to 10 μm; more preferably from 1 μm to 7 μm. According to one embodiment, the d₉₀ granulometry of non-hydrated calcium silicate particles in the mixture is 1, 2, 3, 4, 5, 6, 7, 8, 9, 10, 11, 12, 13, 14, 15, 16, 17, 18, 19 or 20 μm. According to one embodiment, the 4:150 granulometry in the mixture ranges as defined above are determined by laser diffraction.

Calcium Carbonate Granulometry

According to one embodiment, calcium carbonate particles in the mixture have a d₁₀ granulometry ranging from 1 nm to 1000 nm; preferably from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, or from 1 nm to 100 nm. According to one embodiment, calcium carbonate particles in the mixture have a d₁₀ granulometry ranging from 500 nm to 800 nm. According to one embodiment, calcium carbonate particles in the mixture have a d₁₀ granulometry of 600 nm.

According to one embodiment, calcium carbonate particles in the mixture have a 4:150 granulometry ranging from 1 nm to 5 000 nm; preferably from 1 nm to 4000 nm, from 1 nm to 3000 nm, from 1 nm to 2000 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm, or from 1 nm to 100 nm. According to one embodiment, calcium carbonate particles in the mixture have a d₅₀ granulometry ranging from 2500 nm to 3000 nm. According to one embodiment, calcium carbonate particles in the mixture have a d₅₀ granulometry of 2760 nm.

According to one embodiment, calcium carbonate particles in the mixture have a d₉₀ granulometry ranging from 1 nm to 30 000 nm; preferably from 1 nm to 29 000 nm, from 1 nm to 27 000 nm, from 1 nm to 26 000 nm, from 1 nm to 25 000 nm, from 1 nm to 24 000 nm, from 1 nm to 23 000 nm, from 1 nm to 22 000 nm, from 1 nm to 21 000 nm, from 1 nm to 20 000 nm, from 1 nm to 19 000 nm, from 1 nm to 18 000 nm, from 1 nm to 17 000 nm, from 1 nm to 16 000 nm, from 1 nm to 15 000 nm, from 1 nm to 14 000 nm, from 1 nm to 13 000 nm, from 1 nm to 12 000 nm, from 1 nm to 11 000 nm, from 1 nm to 10 000 nm, from 1 nm to 9 000 nm, from 1 nm to 8 000 nm, from 1 nm to 7 000 nm, from 1 nm to 6 000 nm, from 1 nm to 5 000 nm, from 1 nm to 4000 nm, from 1 nm to 3000 nm, from 1 nm to 2000 nm, from 1 nm to 1000 nm, from 1 nm to 900 nm, from 1 nm to 800 nm, from 1 nm to 700 nm, from 1 nm to 600 nm, from 1 nm to 500 nm, from 1 nm to 400 nm, from 1 nm to 300 nm, from 1 nm to 200 nm or from 1 nm to 100 nm. According to one embodiment, calcium carbonate particles in the mixture have a d₉₀ granulometry ranging from 25000 nm to 30000 nm. According to one embodiment, calcium carbonate particles in the mixture have a d₉₀ granulometry of 27400 nm.

Mixing Step—Vibration

In the second method of manufacturing the hydraulic cement of the invention, the mixing step is implemented by a vibration mixer. Without wishing to be bound by any theory, the Applicant submits that the mixing step by vibration provides a self-grinding of at least a part of the calcium carbonate particles contained in the powder mixture, leading to calcium carbonate particles populations having the specific particles sizes distributions of the hydraulic cement of the invention.

According to one embodiment, the mixing step is implemented with a vibration frequency ranging from 1 rpm to 10 000 rpm; preferably ranging from 1000 rpm to 6 000 rpm; more preferably ranging from 3 000 rpm to 5 000 rpm. According to one embodiment, the mixing step is implemented with a vibration frequency of about 1, 100, 200, 300, 400, 500, 600, 700, 800, 900, 1 000, 2 000, 3 000, 4 000, 5 000, 6 000, 7 000, 8 000, 9 000 or 10 000 rpm.

According to one embodiment, the mixing step by vibration is implemented during a vibration time ranging from 1 s to 3600 s; preferably from 1 s to 60 s; more preferably during 30 s. According to one embodiment, the mixing step by vibration is implemented during a vibration time of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 s.

In one embodiment, the mixing step by vibration is performed in presence of an aqueous phase, thereby leading to the composite material of the invention, in a “one pot” method, by a “two-steps-in-one” procedure. During the mixing by vibration, the particles of calcium carbonate and calcium silicate undergo self-grinding (in a much more important measure in the case of the calcium carbonate particles), thereby leading the particles' size distributions of interest. In the same time, the presence of water enables the beginning of the formation of the composite material of the invention. In this embodiment, the aqueous phase may be as defined hereafter with regard to the manufacturing of the composite material.

In one embodiment, the mixing step may include mixing the powder components by gentle mixing in an apparatus providing three-dimensional motion (e.g. Turbula).

Process for Manufacturing the Composite Material

The invention also relates to a method for manufacturing a (hardened) composite material, comprising at least one mixing step of a powder phase with an aqueous phase. According to one embodiment, the method for manufacturing a (hardened) composite material, comprises at least one mixing step of a powder phase comprising or consisting of a hydraulic cement, preferably a hydraulic calcium silicate cement, with an aqueous phase. According to one embodiment, the aqueous phase is an aqueous liquid or an aqueous solution.

According to one embodiment, the method for manufacturing a (hardened) composite material, comprises at least one mixing step of a powder phase comprising calcium silicate particles and calcium carbonate particles, with an aqueous phase. According to one embodiment, the method for manufacturing a (hardened) composite material, comprises at least one mixing step of a powder phase comprising calcium silicate particles, at least one pozzolanic material and calcium carbonate particles, with an aqueous phase. According to one embodiment, the method for manufacturing a (hardened) composite material, comprises at least one mixing step of a powder phase comprising calcium silicate particles, at least one pozzolanic material selected from the group consisting of: fly ash, silica fume, metakaolin, slag, and rice husk ash; and calcium carbonate particles, with an aqueous phase.

According to one embodiment, the powder phase is anhydrous. According to one embodiment, the powder phase is a hydraulic cement. According to one embodiment, the powder phase comprises or consists of a calcium silicate compound, preferably under the form of particles. According to one embodiment, the calcium silicate compound is selected from tricalcium silicate, dicalcium silicate, Portland cement and/or mineral trioxide aggregates (MTA). According to one embodiment, the powder phase is an anhydrous calcium silicate cement powder phase.

First Method

The invention provides a first method for manufacturing a (hardened) composite material, comprises at least one mixing step of a powder phase comprising the hydraulic cement according to the invention, with an aqueous phase. In this method, the mixing step can by performed by any means known by one skilled in the art.

According to one embodiment, the aqueous liquid phase comprises water, preferably purified water.

According to one embodiment, the aqueous liquid phase consists in water. In another embodiment, the aqueous liquid phase is an aqueous solution

According to one embodiment, the aqueous liquid phase comprises from 10 to 100% of water, in weight to the total weight of said aqueous liquid phase, preferably from 20% to 90%, preferably from 30% to 90%, preferably from 35% to 85%. According to one embodiment, the liquid phase comprises from 50% to 90% of water in weight to the total weight of said liquid phase, preferably from 60% to 90%, more preferably from 60% to 85%, more preferably from 65% to 85%.

According to one embodiment, the aqueous phase is an aqueous liquid phase and comprises at least one additive, wherein the additive is preferably selected from setting accelerators and water reducing agents. According to one embodiment, the aqueous liquid phase comprises one or more additives selected from setting accelerators (such as calcium chloride), water reducing agents (such as modified polycarboxylate, glenium, polynaphthalene sulfonate or mixtures thereof) and mixtures thereof.

According to one embodiment, the aqueous liquid phase comprises at least one additive in an amount ranging from 0% to 40% in weight to the total weight of the liquid phase; preferably from 10% to 35%; more preferably from 15% to 35%.

Second Method

The invention also provides a second method for manufacturing a (hardened) composite material, comprising the mixing step by vibration of a powder mixture of calcium silicate and calcium carbonate particles having larger particles sizes than the final targeted particles sizes distributions, with an aqueous phase. This corresponds to the “one pot” method (or “two-steps-in-one” method) mentioned above with regard to the manufacturing of the hydraulic cement of the invention.

The mixing step by vibration may be conducted in the same conditions of vibration frequency and vibration time as detailed above.

Without wishing to be bound by any theory, it seems that the specific features of this second method also afford obtaining a porous intergranular area which porosity is not too high, thus reinforcing the mechanical properties of the composite material, as this is the case by using directly calcium silicate and calcium carbonate particles of particles sizes distributions of interest.

In this second method for manufacturing a (hardened) composite material, the aqueous phase may be as detailed above. The powder phase may comprise calcium silicate, calcium carbonate and additives as detailed above for the second method for manufacturing of the hydraulic cement of the invention.

According to one embodiment, the powder phase comprises calcium silicate particles in an amount ranging from 10% to 100% by weight of the total weight of the powder phase, preferably from 10% to 98%, preferably from 15% to 60%, more preferably from 20 to 55%.

Other Components

According to one embodiment, the powder phase, the aqueous phase and/or the mixture further comprises at least one additive; preferably selected from setting accelerator, radiopacifiers, pigments, pH stabilizing agents, fillers, texturing/thickening agents, water-reducing agents and mixtures thereof. According to one embodiment, the filler is a pozzolanic material; preferably selected from the group consisting of fly ash, silica fume, metakaolin, slag, and rice husk ash.; more preferably is silica fume.

According to one embodiment, the radiopacifier is selected from zirconium oxide, bismuth oxide, cerium oxide, barium sulphate, calcium tungstate, titanate dioxide, ytterbium oxide and mixtures thereof. In a specific embodiment, the radiopacifier is zirconium oxide. According to one embodiment, the setting accelerator is calcium carbonate, calcium oxide, calcium phosphate, sodium bicarbonate, calcium lactate, calcium chloride or mixtures thereof. According to one embodiment, the setting accelerator is calcium carbonate, calcium oxide or mixtures thereof. According to one embodiment, the setting accelerator is calcium chloride. According to one embodiment, the pigments may be iron oxides. According to one embodiment, the water-reducing agent is selected from glenium, polynaphtalene sulfonate, modified polycarboxylate. According to one embodiment, the texturing agents may be for example selected from silica, povidone (also named polyvinylpyrrolidone), cellulose or derivatives thereof such as methylcellulose, hydroxypropylcellulose and hydroxyethylcellulose, polymers such as acrylamide/sodium acryloyldimethyltaurate copolymer isohexadecane and hydroxyethyl acrylate/sodium acryloyl dimethyl taurate copolymer, mineral fillers, fumed silica (hydrophilic and/or hydrophobic), xanthan gum, or mixtures thereof. According to one embodiment, the pH stabilizing agent is a mineral acid or an organic acid. According to one embodiment, the surfactant is a polysorbate.

Powder Phases

According to one embodiment, the powder phase comprises or consists of:
85% of at least one calcium silicate compound, preferably selected from tricalcium silicate, dicalcium silicate and mixtures thereof; and
15% of calcium carbonate; in weight to the total weight of the powder phase.

According to one embodiment, the powder phase comprises or consists of:
50% of at least one calcium silicate compound, preferably selected from tricalcium silicate, dicalcium silicate and mixtures thereof; and
50% of calcium carbonate; in weight to the total weight of the powder phase.

According to one embodiment, the powder phase, the aqueous phase and/or the mixture comprise(s) at least one additive in an amount ranging from 0% to 60% in weight to the total weight of the mixture; preferably from 2% to 50%; more preferably from 2% to 35%. According to one embodiment, the powder phase, the aqueous phase and/or the mixture comprise(s) at least one additive in an amount ranging from 0% to 30% in weight to the total weight of the mixture; preferably from 1% to 25%; more preferably from 1% to 18%.

According to one embodiment, the powder phase, the aqueous phase and/or the mixture comprise(s) from 0 to 40% of radiopacifier in weight to the total weight of said mixture; preferably from 2 to 35%, from 5 to 35%, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35%. According to one embodiment, the powder phase, the aqueous phase and/or the mixture comprise(s) from 0 to 20% of radiopacifier in weight to the total weight of said mixture; preferably from 1 to 18%, from 2 to 18%.

According to one embodiment, the mass ratio of the powder phase to the aqueous phase ranges from 2 to 4.5. According to one embodiment, the mass ratio of the powder phase to the aqueous phase is 2,1; 2,2; 2,3; 2,4; 2,5; 2,6; 2,7; 2,8; 2,9; 3.0; 3.1; 3.2; 3,3; 3,4; 3,5; 3,6; 3,7; 3,8; 3,9; 4.0; 4,1; 4,2; 4,3; 4,4 or 4,5.

According to one embodiment, the powder phase comprises:
calcium silicate particles;
more than 0% to 15% of at least one pozzolanic material; preferably selected from the group consisting of fly ash, silica fume, metakaolin, slag, and rice husk ash; more preferably silica fume; and
more than 0% to 15% of calcium carbonate; in weight to the total weight of the powder phase.

According to one embodiment, the powder phase comprises from:
calcium silicate particles;
more than 0% to 15% of at least one pozzolanic material; preferably selected from the group consisting of fly ash, silica fume, metakaolin, slag, and rice husk ash; more preferably silica fume; and
more than 0% to 15% of calcium carbonate; in weight to the total weight of the mixture.

Uses and Methods for Treating a Subject

Another object of the present invention is the use of the composite material as defined above. According to one embodiment, the (hardened) composite material of the invention is useful as reinforcing material in the non-therapeutical cement field. According to one embodiment, the non-therapeutical cement field is the constructive field. According to one embodiment, the composite material of the invention is for use in the medical field, preferably in the dental or the orthopedic field, as a restorative and/or filling material.

According to one embodiment, the composite materials of the invention may also be used in orthopedics, in bone restoration, in craniofacial and/or maxillofacial surgery.

Another object of the present invention relates to a method for treating the crown of a tooth, for example enamel restoration, permanent dentin restoration, deep or large carious lesions restoration, deep cervical or radicular lesions restoration, pulp capping or pulpotomy; and/or the root of a tooth, such as for example root and furcation perforations, internal/external resorptions, apexification or retrograde surgical filling; in a subject in need thereof, comprising the use of a composite material of the invention as defined above.

According to one embodiment, the composite materials of the invention may be used in treating a bone and/or dental disorder or disease in a subject in need thereof. According to one embodiment, the present invention refers to the use of the composite materials of the invention for treating a bone and/or dental disorder or disease in a subject in need thereof. According to one embodiment, the present invention refers to a method for treating a bone and/or dental disorder or disease in a subject in need thereof by using the composite materials of the invention.

### EXAMPLES

The present invention is further illustrated by the following examples.

### Example 1: Effects of Mixing Parameters on the Granulometry of the Components of the Powder Phase (Hydraulic Calcium Silicate Cement)

The Applicant has studied the effect of the mixing parameters used in the process for manufacturing a hardened cementitious material from a hydraulic calcium silicate cement, on the final granulometry of the components of said cement.

For this goal, the Applicant has studied:
the granulometry of a powder phase A consisting of calcium silicate particles alone,
the granulometry of a powder phase B consisting of calcium carbonate particles alone, and
the granulometry of a powder phase C consisting of 15% of tricalcium silicate particles and 85% of calcium carbonate particles in weight to the total weight of the powder phase.

Each powder phase has then been stirred, without any liquid phase, under vibration with a vibration frequency ranging from 3000 rpm to 3500 rpm, during 30s, i.e. in the mixing conditions usually used to mix the powder phase with the liquid aqueous phase. Then, the d₁₀, d₅₀ and d₉₀ granulometries for each powder phase after mixing have been determined by laser diffraction and compared to their initial d₁₀, d₅₀ and d₉₀ granulometries.

The results are shown in the following Table 1.

TABLE 1

| | d₁₀ | | d₅₀ | | d₉₀ | |
|---|---|---|---|---|---|---|
| | (μm) | | (μm) | | (μm) | |
| | Before | After | Before | After | Before | After |
| Powder | vibra- | vibra- | vibra- | vibra- | vibra- | vibra- |
| phase | tion | tion | tion | tion | tion | tion |
| A | 1.59 | 0.81 | 3.86 | 3.56 | 7.87 | 7.1 |
| B | 0.60 | 0.44 | 2.76 | 1.1 | 27.4 | 3.55 |
| C* | 0.49 | 0.45 | 2.12 | 1.31 | 19.5 | 5.12 |

*determination of the granulometry based on the calcium carbonate refractory index.

The results show that the mixing parameters used in the process for manufacturing the composite material from a hydraulic cement lead to a self-griding of calcium silicate particles and calcium carbonate particles when applied to the powder cement only. Nevertheless, the d₁₀, d₅₀ and d₉₀ granulometries are much more reduced for calcium carbonate particles than for calcium silicate particles.

The self-griding phenomenon is also observed when the calcium silicate and the calcium carbonate particles are mixed together.

### Example 2: Process for Manufacturing a Hardened Composite Material of the Invention

The Applicant has then manufactured hardened cementitious materials from a hydraulic calcium silicate cement which is powder phase C as defined in example 1.

For this aim, the Applicant has mixed the powder phase C with an aqueous liquid phase in mass ratios of the powder phase to the aqueous phase ranging from 2 to 4.5, using a vibration mixer with a vibration frequency ranging from 3000 rpm to 3 500 rpm during 30s.

Surprisingly, even with the low amount (15%) of tricalcium silicate linker present in the powder phase, an efficient setting and a short setting time (around 30 minutes) were obtained with powder phase C.

The obtained materials were observed by microscopy and featured a composite structure comprising:
a solid dispersant phase of calcium silicate hydrates (CSH);
tricalcium silicate particles dispersed in the solid dispersant phase; and
porous intergranular areas located between and around said tricalcium silicate particles; said porous intergranular areas comprising insoluble calcium carbonate particles having a d₁₀, d₅₀ and d₉₀ granulometry reduced compared to the initial d₁₀, d₅₀ and d₉₀ granulometry of calcium carbonate particles introduced in the powder phase.

Thus, the same mechanism of self-griding as evidenced in example 1 on powders occurs upon mixing by vibration the powder phase with the liquid phase to lead to the hardened cement.

### Example 3: Manual Mixing of Grinded Powders

Example 3 shows the advantageous character of the cement's granulometry can be obtained by mixing powders, presenting the adequate granulometry so as to reach the granulometry of the hydraulic cement according to the invention.

Two compositions were prepared by manual mixing according to table 2.

TABLE 2

| | Reference | |
|---|---|---|
| | Composition | |
| Ingredients | (wt. %) | Composition D (wt. %) |
| Micronized Tricalcium silicate | 80.75 | 80.75 |
| Calcium carbonate (Micro) | 14.00 | 10.00 |
| Calcium carbonate (Nano) | | 4.00 |
| Zirconium oxide | 5.00 | 5.00 |
| Calcium oxide | 0.25 | 0.25 |

Wherein 50% of the micronized tricalcium silicate particles presented 50% in volume of the particles has a size of less than 8 μm.

Wherein the granulometry of calcium carbonate (Micro) was as follows:
10% in volume of the particles has a size of less than 0.588 μm,
50% in volume of the particles has a size of less than 2.54 μm, and
40% in volume of the particles have a size ranging from 2.54 μm to 23.96 μm.

Wherein the granulometry of calcium carbonate (Nano) was as follows:
10% in volume of the particles has a size of less than 0.581 μm,
50% in volume of the particles has a size of less than 1.89 μm, and
40% in volume of the particles have a size ranging from 1.89 μm to 8.71 μm.

The resulting calcium carbonate mix in composition D as per in table 2 presented the following granulometry:
10% in volume of the particles has a size of less than 0.586 μm,
50% in volume of the particles has a size of less than 2.35 μm, and
40% in volume of the particles have a size ranging from 2.35 μm to 19.34 μm.

### Example 4: Effects on the Porosity of the Hardened Material Obtained by Manually Mixed Powders

Hardened material was obtained by mixing each of the reference composition and the composition D of example 3 with 190 μL and 202 μL respectively, of a liquid aqueous phase comprising water, calcium chloride and a modified polycarboxylate.

The obtained material was subjected to the assessment of its porosity based on the following protocol:
1. the hardened samples were dried in an oven at 105+/−5° C. for two hours;
2. then the samples were allowed to cool, and weighted (dry weight);
3. the samples were immersed in a beaker containing 50 mL of distilled water, placed under reduced pressure bell jar;
4. the surface of the samples was wiped, and the samples were weighted again (wet weight).

The porosity was then calculated based on the mass ratio of the dry weight to the wet weight. The results are presented in table 3.

TABLE 3

| Samples of | | | | |
|---|---|---|---|---|
| hardened | Dry | Wet | Water | Mean |
| composition | weight | weight | gain | porosity |
| Reference | 0.02621 | 0.05435 | 0.02814 | 0.0662486 |
| | 0.02473 | 0.05247 | 0.02774 | |
| | 0.02447 | 0.05389 | 0.02942 | |
| | 0.02685 | 0.05187 | 0.02502 | |
| | 0.02997 | 0.05591 | 0.02594 | |
| Composition D | 0.03321 | 0.05072 | 0.01751 | 0.0497573 |
| | 0.0321 | 0.05419 | 0.02209 | |
| | 0.03282 | 0.05333 | 0.02051 | |
| | 0.03388 | 0.05498 | 0.0211 | |
| | 0.03706 | 0.05461 | 0.01755 | |

The results show that the granulometry of composition D according to the invention leads to a significant reduction of the hardened material's porosity that is advantageous for its dental applications.

### Example 5: Evaluation of the Compressive Strength of the Hardened Cement

Example 5 shows the advantageous effects of the cement's granulometry can be obtained by mixing fine powders, and then further reducing the particles size by vibration, so as to reach the granulometry of the hydraulic cement according to the invention. Three compositions were prepared according to table 4.

TABLE 4

| | Ref. | | |
|---|---|---|---|
| | Composition | Composition E | Composition F |
| Ingredients | (wt. %) | (wt. %) | (wt. %) |
| Micronized Tricalcium | 80.75 | 50.00 | 50.00 |
| silicate | | | |
| Calcium carbonate | 14.00 | 7.14 | |
| (Micro) | | | |
| Calcium carbonate | | 2.86 | 10.00 |
| (Nano) | | | |
| Zirconium oxide | 5.00 | 40.00 | 40.00 |
| Calcium oxide | 0.25 | | |

Wherein the granulometry of Micronized Tricalcium silicate, Calcium carbonate (Micro), and Calcium carbonate (Nano) were as per in Example 3.

The Reference and the Compositions E and F powdery compositions were then stirred, without any liquid phase, under vibration with a vibration frequency ranging from 3000 rpm to 3500 rpm, during 30s, i.e. in the mixing conditions usually used to mix the powder phase with the liquid aqueous phase.

The amount of Zirconium oxide was increased in compositions E and F because of its abrasive properties that do not impact the applicability of the compositions E and F in dental applications. Indeed, on the Mohs scale, Zirconium oxide hardness is 8-8.5 against 3 for CaCO₃. Calcium carbonate (Nano) was added respecting the same Calcium carbonate (Micro)/Calcium carbonate (Nano) ratio as per in composition D of example 3. It is understood that the mixing parameters in association with the abrasive nature of Zirconium oxide lead to the Calcium carbonate granulometry according to the invention.

### Example 6: Effects on the Mechanical Properties of the Hardened Material Obtained by Mixing Fine Powders Followed by Vibration

The mechanical properties of hardened material obtained from the compositions of example 5 were assessed as follows.

Hardened material test pieces were obtained by mixing each of the reference composition and the compositions E and F of example 5 with 173 μl, 137 μL and 140 μL, respectively, of a liquid aqueous phase comprising water, calcium chloride and a modified polycarboxylate.

The obtained material was subjected to the assessment of its compressive strength based on the following protocol.

The compressive strength is a test carried out on a Universal press bench (model 2/M, MTS Systems, 1400 Eden Prairie, Minneapolis, USA) which measures the maximum force to which the cement can be subjected.

The test consists of the compression by two metal plates of a test piece approximately 5 mm in height and 4 mm in diameter. The maximum stress (N/S) before rupture of the sample is measured. The compression speed was of 0.5 mm/s. The results are presented in table 5.

TABLE 5

| | Hardened | Hardened | Hardened |
|---|---|---|---|
| | Ref. | composition E | composition F |
| Mesure 1 | 291.882 | 270.183 | 314.65 |
| Mesure 2 | 226.956 | 293.331 | 296.5 |
| Mesure 3 | 278.994 | 262.054 | 300.172 |
| Mesure 4 | 277.106 | 276.466 | 300.719 |
| Mesure 5 | 256.235 | 265.493 | 297.725 |
| Mesure 6 | 307.036 | 313.466 | 277.24 |
| Mesure 7 | | 282.116 | 224.258 |
| Mesure 8 | | 318.362 | |
| Mean | 273.0348333 | 285.183875 | 287.3234286 |
| Standard | 28.17401062 | 21.38157868 | 29.89591553 |
| deviation | | | |

The results of example 6 show a significant increase of the compressive strength of test piece obtained from compositions E and F, compared to the test piece obtained from a reference composition. These results highlight the effects on the mechanical properties of the hardened pieces obtained from a composition presenting a fine calcium carbonate particle distribution according to the invention.

1-15. (canceled)

16. A hydraulic cement comprising:
15% to 98% in weight of the total weight of the cement of calcium silicate particles, wherein 50% in volume of the total volume of calcium silicate particles have a size ranging from 1 inn to 10 μm and
0.5% to 85% in weight of the total weight of the cement of calcium carbonate particles, wherein:
10% in volume of the total volume of the calcium carbonate particles have a size of less than 0.59 μm;
50% in volume of the total volume of the calcium carbonate particles have a size of less than 2.5 μm, and
40% in volume of the total volume of the calcium carbonate particles have a size ranging from 2.5 μm to 20 μm.

17. The hydraulic cement according to claim 16, wherein:
10% in volume of the total volume of the calcium carbonate particles have a size of less than 0.55 μm;
50% in volume of the total volume of the calcium carbonate particles have a size of less than 1.5 μm, and
40% in volume of the total volume of the calcium carbonate particles have a size ranging from 1.5 μm to 5.5 μm.

18. The hydraulic cement according to claim 16, wherein the calcium silicate particles are selected from group consisting of tricalcium silicate (C3S), dicalcium silicate (C2S) and any combinations thereof.

19. The hydraulic cement according to claim 16, wherein the calcium silicate particles are in a Portland cement or in a mineral trioxide aggregate (MTA).

20. The hydraulic cement according to claim 16, further comprising at least one additive.

21. The hydraulic cement according to claim 20, wherein the radiopacifier is selected from the group consisting of zirconium oxide, bismuth oxide, cerium oxide, barium sulphate, calcium tungstate, titanate dioxide, ytterbium oxide and mixtures thereof.

22. A method for manufacturing a hydraulic cement according to claim 16, comprising, mixing:
15% to 98% in weight of the total weight of the cement of calcium silicate particles, wherein 50% in volume of the total volume of calcium silicate particles have a size ranging from 1 inn to 10 μm and
0.5% to 85% in weight of the total weight of the cement of calcium carbonate particles, wherein:
10% in volume of the total volume of the calcium carbonate particles have a size of less than 0.59 μm;
50% in volume of the total volume of the calcium carbonate particles have a size of less than 2.5 μm, and
40% in volume of the total volume of the calcium carbonate particles have a size ranging from 2.5 μm to 20 μm.

23. A method for manufacturing a hydraulic cement according to claim 16, comprising:
at least one mixing step of a powder phase comprising:
15% to 98% in weight of the total weight of the cement of calcium silicate particles, wherein 50% in volume of the total volume of calcium silicate particles have a size ranging from 1 inn to 10 μm; and
0.5% to 85% w/w of calcium carbonate particles, wherein:
10% in volume of the total volume of the calcium carbonate particles have a size of less than 1 μm;
50% in volume of the total volume of the calcium carbonate particles have a size of less than 5 μm, and
40% in volume of the total volume of the calcium carbonate particles have a size ranging from 5 μm to 30 μm;
and a simultaneous and/or sequential vibration step leading to the hydraulic cement.

24. The method according to claim 23, wherein the vibration step is implemented with a vibration frequency ranging from 1 rpm to 15,000 rpm; and during a vibration time ranging from 1 s to 3,600 s.

25. The method according to claim 22, wherein the mixing of the powder phase includes a mixing by three-dimensional motion.

26. A method for manufacturing a composite material, comprising at least one mixing step of a hydraulic cement according to claim 16, with an aqueous phase; in a mass ratio of the hydraulic cement to the aqueous phase ranging from 2 to 4.5.

27. A composite material obtained by the process according to claim 26, comprising
a solid dispersant phase comprising or consisting of calcium silicate hydrates (CSH);
calcium silicate particles dispersed in the solid dispersant phase;
a porous intergranular area located between said calcium silicate particles; said porous intergranular area comprising insoluble calcium carbonate particles having a d₅₀ granulometry ranging from 1 nm to 1,500 nm.

28. A kit for producing a composite material, said kit comprising:
a powder phase comprising the hydraulic cement according to claim 16; and
a liquid aqueous phase;
wherein the weight ratio of the powder phase present in the kit to the liquid aqueous phase present in the kit ranges from 2 to 5.

29. A method of manufacturing a composite material in a non-therapeutical cement field, comprising adding as reinforcing material the hydraulic cement according to claim 16 to a composite material for use in the non-therapeutical cement field.

30. The hydraulic cement according to claim 16, for use in the medical field, to form a restorative and/or filling material.

31. The hydraulic cement according to claim 16, wherein the at least one additive is selected from the group consisting of set accelerators, radiopacifiers, pigments, pH stabilizing agents, fillers, texturing/thickening agents, and water-reducing agents.
